(19) United States
(12) Patent Application Publication
(10) Pub. No.: US 2023/0183640 A1
(43) Pub. Date: Jun. 15, 2023

(54) METABOLIC INHIBITORS WITH EFFICACY FOR INHIBITING SULFIDE PRODUCTION IN HARSH ENVIRONMENTS

(71) Applicant: MC (US) 3 LLC, Wilmington, DE (US)

(72) Inventors: Hiteshkumar Dave, Wilmington, DE (US); Gerald O'Connor, Wilmington, DE (US); Eileen Warwick, Wilmington, DE (US)

(21) Appl. No.: 17/925,185

(22) PCT Filed: Apr. 15, 2021
(86) PCT No.: PCT/US2021/027506

§ 371 Date: Nov. 14, 2022

Related U.S. Application Data
(60) Provisional application No. 63/025,540, filed on May 15, 2020.

(51) Int. Cl. C12N 1/38; C09K 8/54; C10G 75/00

(52) U.S. Cl. CPC C12N 1/38; C09K 8/54; C10G 75/00; C09K 2208/20; C10G 2300/207; C12N 2501/999

(57) ABSTRACT

Disclosed herewith is a method of providing N-hydroxycarboxamide compound-based metabolic inhibitor composition, which has demonstrated efficacy for inhibiting sulfide production, under anaerobic conditions. This composition is suitable for use in downhole, drilling and exploration application environments and other harsh environment applications, including mining, industrial extraction of metals and sewage treatment, as well as non-harsh environment applications.

### FIELD OF THE INVENTION

This invention relates to the method of inhibiting sulfide production through contacting a harsh environment with a N-hydroxycarboxamide based metabolic inhibitor composition, with or without biocides.

### BACKGROUND OF THE INVENTION

Sulfide, hydrogen sulfide (H₂S) in particular, generation begins by the introduction of sulfate- or other sulfur-containing aqueous solutions into an anaerobic environment for indigenous microorganisms and microorganisms contained in the aqueous, oil, hydrocarbon containing system or any other system that can produce hydrogen sulfide.

Hydrogen sulfide is toxic, corrosive, and flammable and often causes problems in both the upstream and downstream oil and gas industry. Exposure even at low concentrations, can cause serious injury or death. Considerable expense and effort are expended annually to reduce the H₂S content of gas and oil streams to make them suitable for commercial use. Thus, a need exists for an effective method to inhibit the generation of hydrogen sulfide and reduce the growth of or kill the microbes responsible to produce hydrogen sulfide.

Hydroxamic acids are well known in literature to be useful as histone deacetylase inhibitor drugs with potent antimalarial activity. They have also been reported in literature for use in drugs for their therapeutic potential in treating various tumors and cancers, for example, as described in chapter “Therapeutic Areas II: Cancer, Infectious Diseases, Inflammation & Immunology and Dermatology”, by H. Weinmann, E. Ottow, in Comprehensive Medicinal Chemistry II, 2007.

The article “Synthesis and activities of naphthalimide azoles as a new type of antibacterial and antifungal agents” by Yi-Yi Zhang, Cheng-He Zhou, the use of Naphthalimide derivatives as an antimicrobial agent has been described. The article describes the method of kill of microbes by usage of these derivatives and attributing the effects to the naphthalimide backbone. These compounds may also enhance pharmaceutical properties, indicating the backbone may have the biocidal effects and additions may increase efficacy

The article “Synthesis, Evaluation Antimicrobial Activity of Some New N-substituted Naphthalimides Containing Different Heterocyclic Rings” by Mohammed R. Ahmad, Suaad M. H. Al-Majidi, and Ayad Kareem Khan disclose antibacterial activities of some newly synthesized naphthalimides linked to four or five membered heterocyclic rings against four types of pathogenic bacteria and one type of fungi. The compounds were found to have moderate to high antimicrobial activity.

The article “Antimicrobial activity of N-phthaloylamino acid hydroxamates” by Julija Matijevi-Sosa and Zdenka Cvetnic describes the antibacterial and antifungal activity of N-phthaloylamino acid hydroxamates. It was found that the hydroxamates inhibit growth by chelation of the PDF enzyme metal in both Gram-positive and Gram-negative bacteria, and LpxC enzyme in Gram-negative enzyme. Phthalimides appear to contribute to inhibition by destabilizing m-RNA, while the antifungal activity was not very expressed.

In U.S. Pat. No. 5,279,967A, use of Naphthalimide derivatives in oil and gas industry N,N′-dialkyl-4-amino-1,8-naphthalimides have been disclosed. These compounds have been used to identify and trace hydrocarbons using the fluorescent labeling compounds

U.S. Pat. No. 6,358,746B1 discloses the use of Naphthalimide derivatives in Industrial Water Solutions, for application as a fluorescent tracer in water systems such as in the oil industry.

Both these patents use the derivatives for oil and gas applications however they have not been used in harsh environments to inhibit sulfide production, under anaerobic conditions, in particular.

It has been established that hydroxamate based compounds have antimicrobial activity; however, the disadvantage is that most hydroxamates do not have the stability and efficacy to function in harsh environments. The problem to be solved is to provide a method of providing a composition that can inhibit sulfide production by a sulfide producing organisms, under anaerobic conditions.

### SUMMARY OF THE INVENTION

The present invention is directed to a method of inhibiting sulfide production comprising:
(i) providing a composition comprising at least one compound having structure 1:

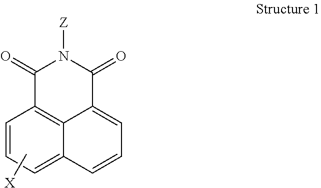

wherein, Z: C(O)NHOH or C(Y)(R),
Y: Hydrogen, C₆ aromatic, C₆ heteroaromatic, C₆ aliphatic cyclic or alicyclic group, hetero group such as nitro, phosphate, hydroxyl,
R: Carbon (n=1-10) linear or branched chain compound terminated with an N-hydroxycarboxamide, carboxylic acid, alcohol or N-hydroxycarboxamide,
X: Hydrogen, OH, NH₂, halogen, carbon (n=1-3) linear or branched chain; compound and
(ii) contacting the composition with a sulfide producing bacteria, under anaerobic conditions, to inhibit the sulfide production.

The present invention is also directed to a method of inhibiting sulfide production comprising:
(i) providing a composition comprising at least one compound having structure 2:

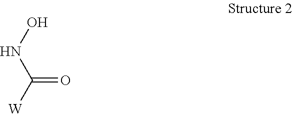

wherein, W is: Hydrogen, carbon (n=1-10) linear or branched chain compound that is optionally terminated with a hydroxyamide, carboxylic acid, alcohol or N-hydroxycarboxamide, C₆ aromatic, C₆ heteroaromatic, C₆ aliphatic cyclic or alicyclic group; and
(ii) contacting the composition with a sulfide producing bacteria, under anaerobic conditions, to inhibit the sulfide production.

### DETAILED DESCRIPTION OF THE INVENTION

These compositions have demonstrated efficacy for inhibiting sulfide production. The compositions are suitable for use in aqueous environments where sulfide exists including downhole, drilling and exploration application oil and gas environments and other harsh environment applications, including mining, industrial extraction of metals and sewage and wastewater treatment and other industrial water and water containing/contaminated systems, as well as non-harsh environment systems.

A number of terms have been used while describing the invention. Unless otherwise specified, the terms are defined as:

As used herein, the articles “a”, “an”, and “the” preceding an element or component of the invention are intended to be nonrestrictive regarding the number of instances (i.e., occurrences) of the element or component. Therefore “a”, “an”, and “the” should be read to include one or at least one, and the singular word form of the element or component also includes the plural unless the number is obviously meant to be singular.

As used herein, the term “comprising” means the presence of the stated features, integers, steps, or components as referred to in the claims, but that it does not preclude the presence or addition of one or more other features, integers, steps, components or groups. The term “comprising” is intended to include embodiments encompassed by the terms “consisting essentially of” and “consisting of”. Similarly, the term “consisting essentially of” is intended to include embodiments encompassed by the term “consisting of”.

As used herein, the term “about” modifying the quantity of an ingredient or reactant employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like.

Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, and the like.

As used herein, Absorbance relates to measure of the capacity of a substance to absorb incident light of a specified wavelength. Absorption is used to quantify specific substances.

As used herein, Aerobic conditions relate to the conditions where microorganisms are growing in presence of oxygen.

As used herein, Anaerobic conditions relate to the conditions where microorganisms are growing in absence of oxygen.

As used herein, Efficacy relates to the ability of tested compounds in inhibiting H₂S.

As used herein, Enumeration plates relate to giving the log growth of a microbial sample by inoculating plates containing fresh media and serial diluting ten-fold. These plates are then incubated for a set amount of time. This helps to determine the number of microorganisms that were present in the original sample.

As used herein, Harsh environment relates to the presence of extreme conditions, for example, extreme high or low temperature, extreme high or low pressure, high or low content of oxygen or carbon dioxide in the atmosphere; high levels of radiation, absence of water; the presence of sulfur, petroleum and natural gases, where it is very hard for life forms to survive. Downhole oil and gas applications is an example of a harsh environment.

As used herein, inhibition of hydrogen sulfide (H₂S) production relates to reducing H₂S levels by greater than or equal to 5%, alternatively greater than or equal to 10%, alternatively greater than or equal to 20%, alternatively greater than or equal to 25%, alternatively greater than or equal to 30% and alternatively greater than or equal to 50% in the harsh environment by either selectively inhibiting sulfate reducing pathways or controlling sulfate reducing bacteria population by effective treatment strategies.

As used herein, Optical density (OD) relates to the measure of absorbance and is defined as the ratio of the intensity of light falling upon a material and the intensity transmitted.

When a parameter is given either as a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. The scope of the invention is not intended to be limited to the specific values and examples as recited in the specification.

The present invention is directed towards methods for inhibiting the reduction reaction of a sulfur-containing compound by a microorganism that produces sulfides in, for example crude oil or hydrocarbon containing systems, which contain greater than or equal to 10 ppm sulfide. This invention highlights the usage of N-hydroxycarboxamide compounds disclosed herewith to inhibit sulfides, H₂S in particular, under anaerobic conditions. This method is useful in oil and gas applications and downhole oilfield reservoirs. This composition could also have applications in non-Oil and Gas applications in inhibiting other problematic bacteria.

Sulfur utilizing prokaryotes can produce hydrogen sulfide through the reduction of sulfate, thiosulfate, sulfite, bisulfite, sulfur, other inorganosulfur compounds, organosulfur compounds, or a combination thereof. The sulfur utilizing prokaryote can comprise a genus or species of bacteria and archaea capable of reducing sulfur compounds to produce a sulfide, hydrogen sulfide or iron sulfide. Preferably, the sulfur utilizing prokaryote can comprise a sulfate-reducing-bacteria. The hydrogen sulfide concentration can be reduced by about 25 to 100 percent, depending on the amount of the composition used and the type of N-hydroxycarboxamide compound used in the composition. Table 2 lists some of the compounds that can be used in the compositions disclosed as embodiments of the invention.

When the method of the present invention of inhibiting sulfide production comprising:
(i) providing a composition comprising at least one compound having structure 1:

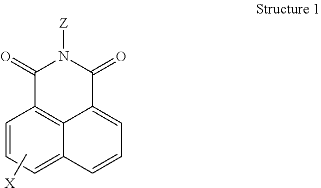

wherein, Z: C(O)NHOH or C(Y)(R),
Y: Hydrogen, C₆ aromatic, C₆ heteroaromatic, C₆ aliphatic cyclic or alicyclic group, hetero group such as nitro, phosphate, hydroxyl,
R: Carbon (n=1-10) linear or branched chain compound terminated with an N-hydroxycarboxamide, carboxylic acid, alcohol or N-hydroxycarboxamide,
X: Hydrogen, OH, NH₂, halogen, carbon (n=1-3) linear or branched chain compound; and

(ii) contacting the composition with a sulfide producing bacteria, under anaerobic conditions, to inhibit the sulfide production.

This composition is preferably

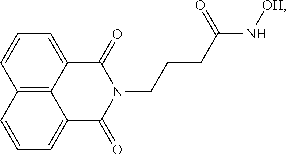 [SMILES: O=C(CCCN1C(=O)c2cccc3cccc(c23)C1=O)NO]

and most preferably comprises

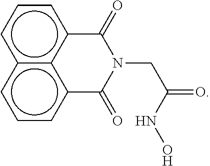

Alternatively, the invention is a method of inhibiting sulfide production comprising:
(i) providing a composition comprising at least one compound having structure 2:

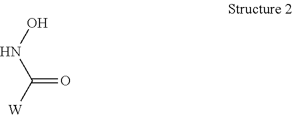

wherein, W: Hydrogen, carbon (n=1-10) linear or branched chain compound that is optionally terminated with a hydroxyamide, carboxylic acid, alcohol or N-hydroxycarboxamide, C₆ aromatic, C₆ heteroaromatic, C₆ aliphatic cyclic or alicyclic group; and
(ii) contacting the composition with a sulfide producing bacteria, under anaerobic conditions, to inhibit the sulfide production.

This composition alsom may comprise:

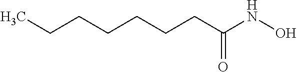

In the method described herein, the compositions are preferably used to inhibit H₂S production in a hydrocarbon containing system, which can be a downhole, a subterranean hydrocarbon-containing formation, a well, a pipeline, a fluid separation vessel, a floating production storage vessel, an offloading vessel, a refinery, or a storage system.

In the method described herein, the compositions can further be administered along with a traditional biocide, or a combination of biocides thereof, for synergistic effects in controlling bacteria.

The compositions can effectively inhibit H₂S in harsh environments like oil and gas downhole applications, subterranean hydrocarbon containing formation, functional fluids, oil and gas reservoirs and production systems, oil and gas transportation and storage systems, mining, industrial extraction of metals etc. This composition can also be effective against problematic bacteria present in non-harsh environments like cooling and heating systems, paper and pulp mills, membrane and filtration systems, as well as in material preservation, gas or liquid produced or used in a waste-water process, farming or slaughter house, land-fill, sewage collection system, municipality waste-water plant, coking coal process, or biofuel process.

### EXAMPLES

### Methods and Analysis

To understand the hydrogen sulfide inhibition efficacy of the N-hydroxycarboxamide compounds of the present invention, several compounds were tested. Some compounds that can be used in this H₂S inhibiting composition are disclosed in Table 2. For the testing procedures, the compounds were dissolved in DMSO, resulting in a stock solution of about 8000-100000 ppm, which was subsequently diluted in 96-well plates or 10 mL serum vials containing media and selected culture to give varying concentrations from 0.09 to 1000 ppm of the compounds in final solution for efficacy testing.

Strains of commonly found bacteria were used for testing the efficacy of the compounds, viz. Desulfovibrio alaskensis, Desulfovibrio longus and Desulfovibrio gabonensis. Media for the cultures was also prepared by using a standard method. The cultures were aseptically used and incubated under anaerobic conditions. Two well established methods were used for testing various compounds:

### (1) 96-Well Plate Method, and

### (2) Serum Test Vial Preparation and Sulfide Assay

The compounds C1, C12, C14, C15 and C16 were tested individually to understand each of their efficacies in inhibiting the H₂S production from sulfate reducing bacteria, under standard temperature and pressure conditions. The efficacy of compounds C14 and C15 against hydrogen sulfide production are disclosed in Example 1, Tables 6 and 7. It can be noted that these compounds did not show significant activity in reducing H₂S production and so, these compounds are not effective in inhibiting H₂S production.

The compound C12 had high efficacy when used in the composition for inhibiting hydrogen sulfide production, under anaerobic conditions. The compound showed efficacy when used in a concentration range of 31.25 to 1000 ppm, preferably in a concentration range of 125 ppm to 1000 ppm. Example 1, Tables 6 and 7 disclose the results of the experiments conducted.

The compound C16 also had high efficacy when used in the composition for inhibiting hydrogen sulfide production, under anaerobic conditions. The compound showed efficacy when used in a concentration range of 125 to 1000 ppm, preferably in a concentration range of 500 ppm to 1000 ppm. Example 1, Tables 6 and 7 disclose the results of the experiments conducted.

Surprisingly, amongst the tested compounds, compound C1 showed the highest efficacy when used in the composition for inhibiting hydrogen sulfide production, under anaerobic conditions. Example 1, Tables 3, 4 and 5 disclose the results of the experiments conducted. The compound showed efficacy when used in a concentration range of 0.2 to 205 ppm, preferably in a concentration range of 1 ppm to 205 ppm and most preferably in a concentration range of 3 ppm to 205 ppm.

Therefore, to further analyze the efficacy of compound C1, comparative testing was done in both aerobic and anaerobic conditions using additional bacteria cultures. For aerobic conditions testing, the following strains were used: Escherichia coli, Pseudomonas aeruginosa, Enterobacter aerogenes, and Klebsiella pneumoniae. For anaerobic testing, the following strains were used: Klebsiella pneumoniae, Enterobacter aerogenes, Escherichia coli, and Enterococcus faecalis.

It was found that, the composition containing compound C1 preferentially inhibited hydrogen sulfide production under anaerobic conditions, as opposed to aerobic conditions. C1 also showed a surprising efficacy in completely killing the various microorganism strains by using metabolic inhibition. These results are disclosed in Examples 2 and 3.

### Example 1: Efficacy of Compounds Against Sulfate Reducing Bacteria (SRB)

Stock Solution Preparation Compounds were purchased from ChemBridge Corporation and Sigma Aldrich. Compounds' stock solutions were prepared by dissolving compounds in dimethyl sulfoxide (DMSO) at 8000 to 100000 PPM concentrations.

TABLE 1
Preparation of ATCC MB1250 media
MB 1250 (pH 7.5)

| Chemical | Amount (g) | Chemical Source |
|---|---|---|
| MgSO₄ | 2.0 | Fisher |
| Na-Citrate | 5.0 | Fisher |
| CaSO₄ × 2H₂O | 1.0 | Fisher |
| NH₄Cl | 1.0 | Fisher |
| K₂HPO₄ | 0.5 | Fisher |
| Na-Lactate | 3.5 | Fisher |
| Yeast Extract | 1.0 | Fisher |
| NaCl | 25.0 | Fisher |
| DI H₂O | 961.0 | |

Media's pH was adjusted to 7.5. 600 μL resazurin and 0.1 g/L Na-thioglycolate (Sigma Aldrich) were added immediately before autoclaving.

### Stock Culture Preparation

A lyophilized Desulfovibrio alaskensis 14563, Desulfovibrio longus 51456, and Desulfovibrio gabonensis 700201 pure cultures received from ATCC were resuspended individually in 500 ul of MB 1250. Aseptically, the content was transferred to a 5-mL tube of MB1250 medium. The cultures were incubated in an anaerobic chamber at 30° C. for 72 hrs. Subsequently, an individual stock culture with a final concentration of 25% glycerol were prepared by adding equal volumes of culture and 50% glycerol. 1 ml of the cultures were then transferred to 2-ml cryogenic vials and stored at −80° C. The purity of the stock cultures was evaluated through PCR, by amplifying the 16S rDNA region, and thus, it was verified that the original ATCC sample was a pure culture. 48-hour cultures of ATCC Desulfovibrio alaskensis 14563, Desulfovibrio longus 51456, and Desulfovibrio gabonensis 700201 were prepared in an anaerobic chamber. Each culture was prepared as a 1:10 culture by taking 1 milliliter (mL) of a pure culture and inoculating 9 milliliters (mL) of fresh MB1250 media. The Desulfovibrio alaskensis 14563 and Desulfovibrio gabonensis 700201 were all grown at 30° C. and the Desulfovibrio longus 51456 culture was grown at 35° C.

### Treatment Preparation

Two well established methods were used for testing various compounds.

(1) 96-Well Plate Method and (2) Serum vial method.

### (1) 96 Well Plate Method (Plate Preparation and Sulfide Assay):

After the 48-hour incubation, 600 microliters (μL) were taken from each culture and optical density (OD) was measured using a Thermofisher Spectronic 200 Spectrophotometer at 600 nm. Each culture was diluted to 0.05 OD₆₀₀ in fresh MB1250 media and added to the 96 well plates. Edge wells were not used due to their inherent variability and evaporation of the media. Each of the components were added at their respective concentrations for a final volume of 500 μL per well. In Table 3, 6 and 7, the concentrations of the compound used for the experiments are listed. Each experiment was done with at least three replicates for different treatments and non-treatment controls. From these test plates, 200 μL were taken and placed into two separate plates for 3 and 6 days. Plates were then sealed with a titer-top and placed into anaerobic boxes and incubated in an anaerobic incubator at 30° C.

At each time point, sulfide samples were taken, and enumeration plates were made. Enumeration plates were made to determine the log growth in each dosed sample. For this process, enumeration plates were prepared by adding 180 μL fresh media MB1250 containing 0.01 wt % ferrous ammonium sulfate. 20 μL was taken from each well of the challenge plates and transferred to the enumeration plates using a 20-200 μL multichannel pipette. Enumeration plates were mixed three times using the multichannel pipette and serial diluted down the plate tenfold (20 μL into 180 μL). This serial dilution process was repeated for all challenge plate rows giving a total of 6 enumeration plates.

After 7 days these enumeration plates were read. The ferrous ammonium sulfate in each plate would be converted to iron sulfide, changing wells with SRB growth from clear to black. By counting the number of black wells in a row, the log growth from the original well in the challenge plate can be determined.

To determine hydrogen sulfide production in each sample, sulfide samples were also taken from each challenge plate. From each well, 9 μL were taken and added to 60 μL of 2% zinc acetate with 0.02% acetic acid. Then, 180 μL of milliQ water was added. 60 μL of stock solution 1 containing 64% sulfuric acid, <1% Dimethyl-4-phenylenediamine (DMPD), water to 100% was added to each well on the plates. This was followed by 3 μL of stock solution 3 containing 50% Iron (III) chloride. All chemicals are ordered from Fisher Scientific and used as received. These were mixed three times and read after 15 minutes. The plates were read at 670 nm using a Biotek microplate reader. Absorbance readings were converted to mM using a standard curve and this was converted to parts per million using the molar mass of sulfur. The standard curves were made using sodium sulfide nonahydrate solutions to give final solutions of 0, 0.125, 0.25 0.5, 1.0, and 1.5 ppm. After reading at 670 nm, plotting this data gives a linear trendline which can be used to determine H₂S level for the samples.

### (2) Serum Test Vial Preparation and Sulfide Assay

After the 48-hour incubation, 600 microliters (μL) were taken from the Desulfovibrio alaskensis 14563 culture and optical density (OD) was measured using a Thermofisher Spectronic 200 Spectrophotometer at 600 nm. Each culture was diluted to 0.05 OD₆₀₀ in fresh MB1250 media and 5 mL of culture solution is transferred to 10 mL Vials and compound Cis stock solution is added. In Table 4 and 5, the concentrations of the compound C1 used for the experiments are listed. Each experiment was done with at least three replicates for different treatments and non-treatment controls. At 2 days, 3 days and 5 days, enumeration plates were made, and sulfide samples were taken in triplicate.

The enumerations were done in triplicate having 20 μL taken from each vial and placing them into 3 wells of first row in a 96-well plate. Then, the same serial dilution process was done with the test plate procedure as described in 96-well plate method. These enumerations were read after 7 days of growth at 30° C. in the anaerobic chamber. For the sulfide assay, 9 μL were taken from each vial and placed in 3 wells with 2% zinc acetate. The assay procedure was the same as described in 96-well plate method.

TABLE 2
List of Representative Compounds.

| Com- | |
|---|---|
| pounds | Structure |
| C1 | 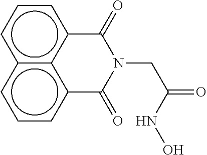 |
| C2 | 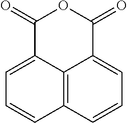 [SMILES: O=C1OC(=O)c2cccc3cccc1c23] |
| C3 | 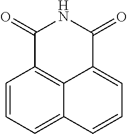 [SMILES: O=C1NC(=O)c2cccc3cccc1c23] |
| C4 | 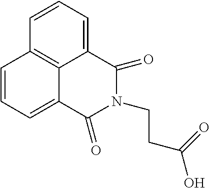 [SMILES: O=C(O)CCN1C(=O)c2cccc3cccc(c23)C1=O] |
| C5 | 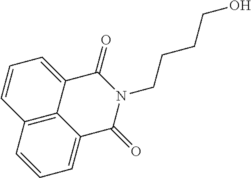 [SMILES: O=C1c2cccc3cccc(c23)C(=O)N1CCCCO] |
| C6 | 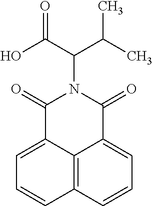 [SMILES: CC(C)C(C(=O)O)N1C(=O)c2cccc3cccc(c23)C1=O] |
| C7 | 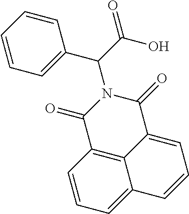 [SMILES: O=C(O)C(c1ccccc1)N1C(=O)c2cccc3cccc(c23)C1=O] |
| C8 | 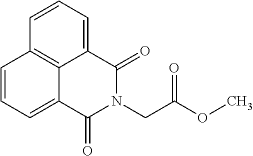 [SMILES: COC(=O)CN1C(=O)c2cccc3cccc(c23)C1=O] |
| C9 | 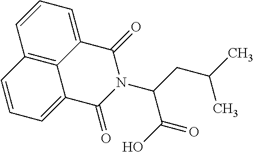 [SMILES: CC(C)CC(C(=O)O)N1C(=O)c2cccc3cccc(c23)C1=O] |
| C10 | 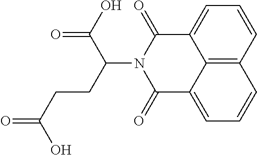 [SMILES: O=C(O)CCC(C(=O)O)N1C(=O)c2cccc3cccc(c23)C1=O] |
| C11 | 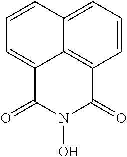 [SMILES: O=C1c2cccc3cccc(c23)C(=O)N1O] |
| C12 | 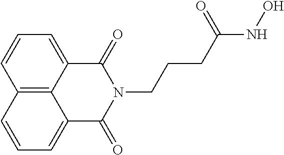 [SMILES: O=C(CCCN1C(=O)c2cccc3cccc(c23)C1=O)NO] |
| C13 | 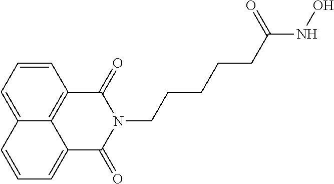 [SMILES: O=C(CCCCCN1C(=O)c2cccc3cccc(c23)C1=O)NO] |
| C14 | 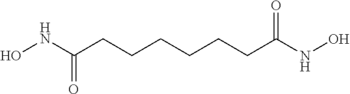 [SMILES: O=C(CCCCCCC(=O)NO)NO] |
| C15 | 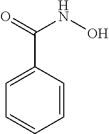 |
| C16 | 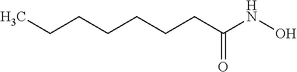 |

TABLE 3
H₂S and Log (Growth) efficacy of compound
C1 against SRBs using 96-Well Plate Method.

| | | Com- | | | | |
|---|---|---|---|---|---|---|
| | Testing | pound | | | | |
| | Time | Conc. | H₂S | Std. | Log | Std. |
| Organisms | (Days) | (PPM) | (PPM) | Dev. | (Growth) | Dev. |
| Desulfovibrio | 3 days | 205.0 | 10.0 | 0.4 | 0.0 | 0.0 |
| gabonensis | | 102.5 | 10.5 | 0.5 | 0.0 | 0.0 |
| 700201 | | 51.3 | 12.3 | 0.7 | 0.0 | 0.0 |
| | | 25.6 | 11.1 | 1.3 | 0.0 | 0.0 |
| | | 12.8 | 12.1 | 0.9 | 0.0 | 0.0 |
| | | 6.4 | 12.4 | 1.0 | 0.0 | 0.0 |
| | | 0.0 | 212.8 | 11.9 | 8.0 | 0.0 |
| Desulfovibrio | 3 days | 205.0 | 8.7 | 0.4 | 0.0 | 0.0 |
| longus | | 102.5 | 8.6 | 0.1 | 0.0 | 0.0 |
| 51456 | | 51.3 | 8.8 | 0.2 | 0.0 | 0.0 |
| | | 25.6 | 9.1 | 0.3 | 0.0 | 0.0 |
| | | 12.8 | 9.3 | 0.5 | 0.0 | 0.0 |
| | | 6.4 | 9.8 | 0.3 | 0.0 | 0.0 |
| | | 0.0 | 263.8 | 24.9 | 8.0 | 0.0 |
| Desulfovibrio | 3 days | 205.0 | 12.7 | 1.0 | 0.3 | 0.6 |
| alaskensis | | 102.5 | 13.6 | 0.8 | 0.3 | 0.6 |
| 14563 | | 51.3 | 15.4 | 0.3 | 0.0 | 0.0 |
| | | 25.6 | 15.1 | 1.0 | 0.0 | 0.0 |
| | | 12.8 | 13.5 | 1.1 | 2.0 | 1.0 |
| | | 6.4 | 15.5 | 0.7 | 2.7 | 2.5 |
| | | 0.0 | 167.1 | 36.6 | 8.0 | 0.0 |
| Desulfovibrio | 6 days | 205.0 | 8.5 | 0.7 | 0.0 | 0.0 |
| gabonensis | | 102.5 | 8.6 | 0.6 | 0.0 | 0.0 |
| 700201 | | 51.3 | 8.4 | 0.3 | 0.0 | 0.0 |
| | | 25.6 | 9.4 | 0.8 | 0.0 | 0.0 |
| | | 12.8 | 10.4 | 0.4 | 0.0 | 0.0 |
| | | 6.4 | 18.1 | 2.3 | 0.0 | 0.0 |
| | | 0.0 | 97.0 | 14.6 | 8.0 | 0.0 |
| Desulfovibrio | 6 days | 205.0 | 8.1 | 0.5 | 0.0 | 0.0 |
| longus | | 102.5 | 7.5 | 0.3 | 0.0 | 0.0 |
| 51456 | | 51.3 | 7.2 | 0.3 | 0.0 | 0.0 |
| | | 25.6 | 7.3 | 0.2 | 0.0 | 0.0 |
| | | 12.8 | 7.9 | 0.6 | 0.0 | 0.0 |
| | | 6.4 | 8.1 | 0.2 | 0.0 | 0.0 |
| | | 0.0 | 190.8 | 28.7 | 7.7 | 0.6 |
| Desulfovibrio | 6 days | 205.0 | 9.0 | 0.6 | 0.0 | 0.0 |
| alaskensis | | 102.5 | 8.7 | 0.6 | 0.0 | 0.0 |
| 14563 | | 51.3 | 8.3 | 0.4 | 0.0 | 0.0 |
| | | 25.6 | 8.8 | 0.4 | 1.0 | 1.7 |
| | | 12.8 | 23.5 | 22.8 | 2.7 | 4.6 |
| | | 6.4 | 18.1 | 14.6 | 3.3 | 4.0 |
| | | 0.0 | 44.8 | 10.1 | 8.0 | 0.0 |

TABLE 4
H₂S and Log (Growth) efficacy of compound C1 against
Desulfovibrio alaskensis 14563 using 10 mL Serum Vial Method.

| Testing | Compound | | | | |
|---|---|---|---|---|---|
| Time | Conc. | H₂S | | Log | |
| (Days) | (PPM) | (PPM) | Std. Dev. | (Growth) | Std. Dev. |
| 3 days | 6.0 | 19.2 | 0.9 | 0.0 | 0.0 |
| | 3.0 | 23.1 | 1.4 | 4.0 | 1.8 |
| | 1.5 | 50.1 | 3.4 | 4.8 | 0.7 |
| | 0.75 | 190.2 | 8.6 | 7.4 | 0.5 |
| | 0 | NA** | NA** | 8.0 | 0.0 |
| 5 days | 6.0 | 15 | 1.0 | 0.0 | 0.0 |
| | 3.0 | 19.5 | 1.4 | 1.0 | 1.3 |
| | 1.5 | 69.9 | 14.7 | 6.2 | 1.0 |
| | 0.75 | 253.3 | 9.0 | 7.9 | 0.3 |
| | 0 | 430.8 | 8.4 | 7.9 | 0.3 |

**Signal overflow during H₂S measurement.

TABLE 5
H₂S and Log (Growth) efficacy of compound C1 against
Desulfovibrio alaskensis 14563 using 10 mL Serum Vial Method.

| Testing | Compound | | | | |
|---|---|---|---|---|---|
| Time | Conc. | H₂S | | Log | |
| (Days) | (PPM) | (PPM) | Std. Dev. | (Growth) | Std. Dev. |
| 2 days | 0.75 | 121.6 | 11.1 | 7.2 | 0.4 |
| | 0.38 | 170.4 | 9.6 | 7.8 | 0.4 |
| | 0.19 | 202.1 | 11.0 | 8.0 | 0.0 |
| | 0.09 | 233.1 | 13.2 | 7.9 | 0.3 |
| | 0.00 | 236.3 | 9.6 | 7.8 | 0.4 |

TABLE 6
H₂S and Log (Growth) efficacy of compounds against
Desulfovibrio alaskensis 14563 using 96-Well Plate Method.

| | Testing | Compound | | | | |
|---|---|---|---|---|---|---|
| | Time | Conc. | H₂S | Std. | Log | Std. |
| Compound | (Days) | (PPM) | (PPM) | Dev. | (Growth) | Dev. |
| C12 | 2 days | 1000 | 5.4 | 0.3 | 0.0 | 0.0 |
| | | 500 | 8.7 | 1.6 | 0.0 | 0.0 |
| | | 125 | 91.4 | 2.0 | 7.0 | 0.0 |
| | | 31.25 | 90.1 | 5.2 | 7.0 | 0.0 |
| | | 15.75 | 115.3 | 0.8 | 7.5 | 0.7 |
| | | 7.615 | 77.6 | 15.2 | 7 | 0.0 |
| | | 0 | 88.4 | 41.7 | 7.5 | 0.6 |
| C14 | 2 days | 1000 | 117.5 | 17.5 | 7.7 | 0.6 |
| | | 500 | 54.6 | 3.6 | 7.7 | 0.6 |
| | | 125 | 131.9 | 6.5 | 8.0 | 0.0 |
| | | 31.25 | 56.3 | 5.8 | 7.3 | 0.6 |
| | | 15.75 | 56.4 | 2.3 | 8.0 | 0.0 |
| | | 7.615 | 71.3 | 28.9 | 7.0 | 0.0 |
| | | 0 | 88.4 | 41.7 | 7.5 | 0.5 |
| C15 | 2 days | 1000 | 130.7 | 26.6 | 8.0 | 0.0 |
| | | 500 | 155.7 | 80.9 | 7.3 | 0.6 |
| | | 125 | 130.2 | 18.2 | 7.3 | 0.6 |
| | | 31.25 | 54.4 | 4.7 | 7.0 | 0.0 |
| | | 15.75 | 54.7 | 4.0 | 7.7 | 0.6 |
| | | 7.615 | 103.6 | 2.0 | 7.7 | 0.6 |
| | | 0 | 88.4 | 41.7 | 7.5 | 0.5 |
| C12 | 5 days | 1000 | 4.8 | 0.3 | 0.0 | 0.0 |
| | | 500 | 6.1 | 0.0 | 0.0 | 0.0 |
| | | 125 | 21.3 | 3.8 | 3.0 | 0.0 |
| | | 31.25 | 42.5 | 0.8 | 7.0 | 0.0 |
| | | 15.75 | 26.7 | 0.1 | 8.0 | 0.0 |
| | | 7.615 | 17.5 | 4.3 | 8.0 | 0.0 |
| | | 0 | 47.3 | 14.4 | 7.7 | 0.5 |
| C14 | 5 days | 1000 | 38.6 | 2.9 | 8.0 | 0.0 |
| | | 500 | 42.2 | 7.4 | 7.0 | 0.0 |
| | | 125 | 45.0 | 6.8 | 7.3 | 1.2 |
| | | 31.25 | 29.0 | 3.4 | 7.3 | 0.6 |
| | | 15.75 | 34.7 | 2.3 | 7.3 | 0.6 |
| | | 7.615 | 45.6 | 2.6 | 7.3 | 0.6 |
| | | 0 | 47.3 | 14.4 | 7.7 | 0.5 |
| C15 | 5 days | 1000 | 73.3 | 6.1 | 8.0 | 0.0 |
| | | 500 | 31.8 | 10.6 | 7.0 | 1.0 |
| | | 125 | 55.5 | 7.8 | 7.0 | 0.0 |
| | | 31.25 | 26.3 | 2.1 | 7.7 | 0.6 |
| | | 15.75 | 42.7 | 18.3 | 7.3 | 0.6 |
| | | 7.615 | 35.2 | 12.4 | 8.0 | 0.0 |
| | | 0 | 47.3 | 14.4 | 7.7 | 0.5 |

TABLE 7
H₂S and Log (Growth) efficacy of compounds against
Desulfovibrio alaskensis 14563 using 96-Well Plate Method.

| | Testing | Compound | | | | |
|---|---|---|---|---|---|---|
| | Time | Conc. | H₂S | Std. | Log | Std. |
| Compound | (Days) | (PPM) | (PPM) | Dev. | (Growth) | Dev. |
| C16 | 2 days | 1000 | 2.2 | 0.2 | 3.0 | 2.6 |
| | | 500 | 2.7 | 0.2 | 5.3 | 0.6 |
| | | 125 | 13.3 | 11.9 | 6.3 | 0.6 |
| | | 31.25 | 35.1 | 15.3 | 7.3 | 0.6 |
| | | 15.75 | 76.9 | 14.9 | 7.7 | 0.6 |
| | | 7.615 | 83.5 | 18.2 | 7.7 | 0.6 |
| | | 0 | 47.5 | 21.0 | 7.7 | 0.5 |
| C12 | 2 days | 1000 | 1.8 | 0.0 | 2.5 | 0.7 |
| | | 500 | 2.2 | 0.0 | 3.0 | 0.0 |
| | | 125 | 12.2 | 0.3 | 5.0 | 1.4 |
| | | 31.25 | 18.2 | 4.0 | 7.5 | 0.7 |
| | | 15.75 | 43.7 | 12.3 | 7.3 | 0.6 |
| | | 7.615 | 67.5 | 0.9 | 8.0 | 0.0 |
| | | 0 | 47.5 | 21.0 | 7.7 | 0.5 |
| C16 | 5 days | 1000 | 2.0 | 0.2 | 0.0 | 0.0 |
| | | 500 | 2.1 | 0.4 | 4.3 | 0.6 |
| | | 125 | 6.9 | 5.8 | 5.0 | 0.0 |
| | | 31.25 | 22.1 | 11.6 | 7.3 | 0.6 |
| | | 15.75 | 29.2 | 8.7 | 7.0 | 0.0 |
| | | 7.615 | 27.7 | 7.7 | 7.3 | 0.6 |
| | | 0 | 23.4 | 3.0 | 7.5 | 0.5 |
| C12 | 5 days | 1000 | 2.0 | 0.0 | 0.0 | 0.0 |
| | | 500 | 1.9 | 0.0 | 0.0 | 0.0 |
| | | 125 | 6.2 | 0.9 | 5.0 | 1.4 |
| | | 31.25 | 15.1 | 0.6 | 5.5 | 0.7 |
| | | 15.75 | 34.4 | 0.4 | 6.7 | 0.6 |
| | | 7.615 | 30.6 | 3.8 | 7.3 | 0.6 |
| | | 0 | 23.4 | 3.0 | 7.5 | 0.5 |

### Example 2: Efficacy of Compound C1 Against Various Non-SRB Bacteria in Aerobic Conditions

### Preparation of Tryptic Soy Broth (TSB) Media

Tryptic soy broth was prepared by dissolving 30 grams of BD Bacto Tryptic Soy Broth powder (ordered from Fisher Scientific) into 1 liter of deionized water. This was autoclaved in a liquid 30 cycle. The Phosphate buffer used was Hardy Diagnostics Dilu-Lok Dilution Vials and received from Fisher Scientific.

### Aerobic Culture Preparation

24-hour cultures were made of ATCC Escherichia coli 8739, Pseudomonas aeruginosa 15442, Enterobacter aerogenes 13048, and Klebsiella pneumoniae 13883. The cultures were prepared by taking a loop of a pure bacterial colony and inoculating 10 mL of TSB. These were grown for 24 hours at 30° C.

### Test Plate Preparation

Following the 24-hour incubation, 600 microliters (μL) were taken from each culture and optical density (OD) was measured using a Thermofisher Spectronic 200 Spectrophotometer at 600 nm. Each culture was diluted to 0.05 OD₆₀₀ in fresh Phosphate Buffered Saline (PBS). Using these cultures, two deep well plates were prepared. Edge wells were not used due to their inherent variability and evaporation of the media. Each of the components were added at their respective concentrations for a final volume of 5004 per well. In Table 8, the concentrations of the compound C1 used for the experiments are listed. Each experiment was done with at least three replicates for different treatments and non-treatment controls. Plates were then sealed with a titer-top and incubated in an incubator at 30° C.

### Enumerations

Enumerations were done at each time point, which included 0 hour, 1 hour, 4 hours and 24 hours. Enumeration plates were made to determine the log growth in each dosed sample. For this process, enumeration plates were prepared by adding 180 μL fresh TSB. 20 μL was taken from each well of the challenge plates and transferred to the enumeration plates using a 20-200 μL multichannel pipette. Enumeration plates were mixed three times using the multichannel pipette and serial diluted down the plate tenfold (20 μL into 180 μL). This serial dilution process was repeated for all challenge plate rows giving a total of 6 enumeration plates. These enumeration plates were read after 24-hours and by counting the number of turbid wells in a row.

TABLE 8
Log (Growth) efficacy of compound C1 in Aerobic
Conditions against various non-SRB bacteria.

| | Testing | Compound | | |
|---|---|---|---|---|
| | Time | Conc. | Log | Std. |
| Organisms | (hours) | (PPM) | (Growth) | Dev. |
| Escherichia coli | 0 | 200 | 7.000 | 1.000 |
| 8739 | | 100 | 7.000 | 0.000 |
| | | 50 | 7.333 | 0.577 |
| | | 25 | 7.000 | 1.000 |
| | | 12.5 | 6.667 | 0.577 |
| | | 6.25 | 7.000 | 0.000 |
| | | 0 | 7.000 | 1.000 |
| Pseudomonas aeruginosa | 0 | 200 | 7.667 | 0.577 |
| 15442 | | 100 | 8.000 | 0.000 |
| | | 50 | 7.667 | 0.577 |
| | | 25 | 7.333 | 0.577 |
| | | 12.5 | 7.667 | 0.577 |
| | | 6.25 | 7.333 | 0.577 |
| | | 0 | 8.000 | 0.000 |
| Enterobacter aerogenes | 0 | 200 | 7.667 | 0.577 |
| 13048 | | 100 | 7.000 | 1.000 |
| | | 50 | 7.667 | 0.577 |
| | | 25 | 7.333 | 0.577 |
| | | 12.5 | 8.000 | 0.000 |
| | | 6.25 | 7.667 | 0.577 |
| | | 0 | 7.333 | 0.577 |
| Klebsiella pneumoniae | 0 | 200 | 6.333 | 0.577 |
| 13883 | | 100 | 6.667 | 0.577 |
| | | 50 | 6.667 | 0.577 |
| | | 25 | 6.667 | 0.577 |
| | | 12.5 | 6.333 | 0.577 |
| | | 6.25 | 7.667 | 0.577 |
| | | 0 | 7.000 | 0.000 |
| Escherichia coli | 1 | 200 | 6.333 | 0.577 |
| 8739 | | 100 | 6.333 | 0.577 |
| | | 50 | 6.333 | 0.577 |
| | | 25 | 7.333 | 0.577 |
| | | 12.5 | 8.000 | 0.000 |
| | | 6.25 | 7.000 | 0.000 |
| | | 0 | 6.667 | 0.577 |
| Pseudomonas aeruginosa | 1 | 200 | 7.000 | 0.000 |
| 15442 | | 100 | 8.000 | 0.000 |
| | | 50 | 7.333 | 1.155 |
| | | 25 | 7.667 | 0.577 |
| | | 12.5 | 8.000 | 0.000 |
| | | 6.25 | 7.000 | 0.000 |
| | | 0 | 7.000 | 1.000 |
| Enterobacter aerogenes | 1 | 200 | 8.000 | 0.000 |
| 13048 | | 100 | 7.333 | 0.577 |
| | | 50 | 7.000 | 0.000 |
| | | 25 | 7.333 | 0.577 |
| | | 12.5 | 8.000 | 0.000 |
| | | 6.25 | 7.667 | 0.577 |
| | | 0 | 7.000 | 0.000 |
| Klebsiella pneumoniae | 1 | 200 | 8.000 | 0.000 |
| 13883 | | 100 | 7.333 | 0.577 |
| | | 50 | 6.000 | 0.000 |
| | | 25 | 6.333 | 0.577 |
| | | 12.5 | 8.000 | 0.000 |
| | | 6.25 | 6.333 | 0.577 |
| | | 0 | 7.333 | 0.577 |
| Escherichia coli | 4 | 200 | 6.667 | 0.577 |
| 8739 | | 100 | 7.000 | 0.000 |
| | | 50 | 6.667 | 0.577 |
| | | 25 | 7.000 | 0.000 |
| | | 12.5 | 7.333 | 0.577 |
| | | 6.25 | 7.000 | 0.000 |
| | | 0 | 6.333 | 0.577 |
| Pseudomonas aeruginosa | 4 | 200 | 7.000 | 1.000 |
| 15442 | | 100 | 7.667 | 0.577 |
| | | 50 | 7.000 | 1.000 |
| | | 25 | 7.000 | 0.000 |
| | | 12.5 | 8.000 | 0.000 |
| | | 6.25 | 7.000 | 0.000 |
| | | 0 | 8.000 | 0.000 |
| Enterobacter aerogenes | 4 | 200 | 7.000 | 0.000 |
| 13048 | | 100 | 8.000 | 0.000 |
| | | 50 | 7.333 | 0.577 |
| | | 25 | 7.667 | 0.577 |
| | | 12.5 | 7.667 | 0.577 |
| | | 6.25 | 7.667 | 0.577 |
| | | 0 | 7.667 | 0.577 |
| Klebsiella pneumoniae | 4 | 200 | 7.000 | 0.000 |
| 13883 | | 100 | 7.000 | 0.000 |
| | | 50 | 6.667 | 0.577 |
| | | 25 | 6.667 | 0.577 |
| | | 12.5 | 6.667 | 0.577 |
| | | 6.25 | 7.667 | 0.577 |
| | | 0 | 7.333 | 0.577 |
| Escherichia coli | 24 | 200 | 7.000 | 0.000 |
| 8739 | | 100 | 6.667 | 0.577 |
| | | 50 | 7.000 | 0.000 |
| | | 25 | 6.667 | 0.577 |
| | | 12.5 | 7.333 | 0.577 |
| | | 6.25 | 6.333 | 0.577 |
| | | 0 | 7.333 | 0.577 |
| Pseudomonas aeruginosa | 24 | 200 | 7.667 | 0.577 |
| 15442 | | 100 | 8.000 | 0.000 |
| | | 50 | 7.667 | 0.577 |
| | | 25 | 7.333 | 0.577 |
| | | 12.5 | 7.000 | 0.000 |
| | | 6.25 | 7.333 | 0.577 |
| | | 0 | 8.000 | 0.000 |
| Enterobacter aerogenes | 24 | 200 | 7.333 | 0.577 |
| 13048 | | 100 | 7.000 | 0.000 |
| | | 50 | 6.667 | 0.577 |
| | | 25 | 7.000 | 0.000 |
| | | 12.5 | 7.333 | 0.577 |
| | | 6.25 | 6.667 | 0.577 |
| | | 0 | 7.667 | 0.577 |
| Klebsiella pneumoniae | 24 | 200 | 7.000 | 1.000 |
| 13883 | | 100 | 7.000 | 1.000 |
| | | 50 | 6.000 | 0.000 |
| | | 25 | 6.000 | 0.000 |
| | | 12.5 | 7.333 | 0.577 |
| | | 6.25 | 7.333 | 0.577 |
| | | 0 | 7.333 | 0.577 |

### Example 3: Efficacy of Compound C1 Against Various Non-SRB Bacteria in Anaerobic Conditions

### Stock Culture Preparation

Klebsiella pneumoniae 13883, Enterobacter aerogenes 13048, Escherichia coli 8739, and Enterococcus faecalis 29212 cultures were made by adding one loop from a freezer stock to 10 mL of fresh Phenol Red Media. These were grown anaerobically at 30° C. for 24 hours.

### Test Plate Preparation

Following the 24-hour incubation, each culture was diluted to 1:10 in fresh Phenol Red Media. Using these cultures, two deep well plates were prepared. Edge wells were not used due to their inherent variability and evaporation of the media. Each of the components were added at their respective concentrations for a final volume of 250 μL per well. In Table 9, the concentrations of the compound C1 used for the experiments are listed. Each experiment was done with at least three replicates for different treatments and non-treatment controls. Plates were then sealed with a titer-top and incubated at room temperature in anaerobic chamber. Enumerations were conducted at 0 hour, 1 hour, 4 hours, and 24 hours. The process for these enumerations was the same as that done for example 2; however, utilizing phenol red media instead of tryptic soy broth.

TABLE 9
Log (Growth) efficacy of compound C1 in various
non-SRB bacteria in anaerobic conditions.

| | Testing | Compound | | |
|---|---|---|---|---|
| | Time | Conc. | Log | |
| Organisms | (hours) | (PPM) | (Growth) | Std. Dev. |
| Klebsiella pneumoniae | 0 | 500 | 6.000 | 0.000 |
| 13883 | | 250 | 6.000 | 0.000 |
| | | 125 | 6.667 | 1.155 |
| | | 62.5 | 6.667 | 0.577 |
| | | 31.25 | 6.667 | 0.577 |
| | | 15.625 | 6.667 | 0.577 |
| | | 0 | 7.000 | 1.000 |
| Enterobacter aerogenes | 0 | 500 | 6.333 | 0.577 |
| 13048 | | 250 | 6.667 | 0.577 |
| | | 125 | 6.333 | 0.577 |
| | | 62.5 | 6.333 | 0.577 |
| | | 31.25 | 6.667 | 0.577 |
| | | 15.625 | 6.333 | 0.577 |
| | | 0 | 6.667 | 0.577 |
| Escherichia coli 8739 | 0 | 500 | 6.333 | 0.577 |
| | | 250 | 6.000 | 0.000 |
| | | 125 | 6.333 | 0.577 |
| | | 62.5 | 6.667 | 0.577 |
| | | 31.25 | 6.667 | 0.577 |
| | | 15.625 | 7.000 | 0.000 |
| | | 0 | 6.667 | 1.155 |
| Enterococcus faecalis | 0 | 500 | 6.667 | 0.577 |
| 29212 | | 250 | 7.000 | 0.000 |
| | | 125 | 6.333 | 0.577 |
| | | 62.5 | 7.000 | 0.000 |
| | | 31.25 | 6.667 | 0.577 |
| | | 15.625 | 7.000 | 0.000 |
| | | 0 | 7.000 | 0.000 |
| Klebsiella pneumoniae | 1 | 500 | 4.333 | 0.577 |
| 13883 | | 250 | 4.333 | 0.577 |
| | | 125 | 3.667 | 0.577 |
| | | 62.5 | 5.000 | 0.000 |
| | | 31.25 | 5.667 | 0.577 |
| | | 15.625 | 6.667 | 0.577 |
| | | 0 | 6.000 | 0.000 |
| Enterobacter aerogenes | 1 | 500 | 5.667 | 1.155 |
| 13048 | | 250 | 5.667 | 0.577 |
| | | 125 | 6.000 | 0.000 |
| | | 62.5 | 7.000 | 0.000 |
| | | 31.25 | 6.333 | 0.577 |
| | | 15.625 | 7.000 | 0.000 |
| | | 0 | 6.000 | 0.000 |
| Escherichia coli 8739 | 1 | 500 | 6.000 | 1.000 |
| | | 250 | 6.000 | 0.000 |
| | | 125 | 6.000 | 0.000 |
| | | 62.5 | 6.333 | 0.577 |
| | | 31.25 | 6.000 | 1.000 |
| | | 15.625 | 7.000 | 0.000 |
| | | 0 | 6.667 | 0.577 |
| Enterococcus faecalis | 1 | 500 | 6.000 | 0.000 |
| 29212 | | 250 | 6.333 | 0.577 |
| | | 125 | 5.667 | 0.577 |
| | | 62.5 | 6.333 | 0.577 |
| | | 31.25 | 6.000 | 0.000 |
| | | 15.625 | 6.667 | 0.577 |
| | | 0 | 7.000 | 0.000 |
| Klebsiella pneumoniae | 4 | 500 | 2.000 | 0.000 |
| 13883 | | 250 | 2.333 | 0.577 |
| | | 125 | 1.667 | 0.577 |
| | | 62.5 | 2.000 | 0.000 |
| | | 31.25 | 2.000 | 1.000 |
| | | 15.625 | 3.333 | 0.577 |
| | | 0 | 8.000 | 0.000 |
| Enterobacter aerogenes | 4 | 500 | 5.333 | 0.577 |
| 13048 | | 250 | 5.333 | 0.577 |
| | | 125 | 5.000 | 0.000 |
| | | 62.5 | 4.667 | 0.577 |
| | | 31.25 | 7.000 | 1.000 |
| | | 15.625 | 7.333 | 0.577 |
| | | 0 | 7.000 | 0.000 |
| Escherichi coli 8739 | 4 | 500 | 4.333 | 0.577 |
| | | 250 | 5.000 | 1.000 |
| | | 125 | 4.667 | 0.577 |
| | | 62.5 | 5.333 | 0.577 |
| | | 31.25 | 6.667 | 1.528 |
| | | 15.625 | 6.667 | 1.155 |
| | | 0 | 7.000 | 0.000 |
| Enterococcus faecalis | 4 | 500 | 4.667 | 0.577 |
| 29212 | | 250 | 4.333 | 0.577 |
| | | 125 | 4.333 | 0.577 |
| | | 62.5 | 4.667 | 0.577 |
| | | 31.25 | 5.667 | 0.577 |
| | | 15.625 | 6.667 | 0.577 |
| | | 0 | 7.333 | 0.577 |
| Klebsiella pneumoniae | 24 | 500 | 0.000 | 0.000 |
| 13883 | | 250 | 0.000 | 0.000 |
| | | 125 | 1.667 | 2.887 |
| | | 62.5 | 5.667 | 0.577 |
| | | 31.25 | 4.667 | 4.163 |
| | | 15.625 | 6.333 | 0.577 |
| | | 0 | 7.667 | 0.577 |
| Enterobacter aerogenes | 24 | 500 | 6.000 | 1.000 |
| 13048 | | 250 | 6.333 | 0.577 |
| | | 125 | 6.000 | 0.000 |
| | | 62.5 | 6.333 | 0.577 |
| | | 31.25 | 6.667 | 0.577 |
| | | 15.625 | 7.667 | 0.577 |
| | | 0 | 7.667 | 0.577 |
| Escherichia coli 8739 | 24 | 500 | 2.667 | 1.155 |
| | | 250 | 4.000 | 0.000 |
| | | 125 | 3.000 | 0.000 |
| | | 62.5 | 2.667 | 0.577 |
| | | 31.25 | 4.000 | 1.000 |
| | | 15.625 | 6.000 | 1.000 |
| | | 0 | 7.000 | 0.000 |
| Enterococcus faecalis | 24 | 500 | 4.000 | 1.000 |
| 29212 | | 250 | 4.333 | 0.577 |
| | | 125 | 5.667 | 0.577 |
| | | 62.5 | 5.667 | 0.577 |
| | | 31.25 | 5.333 | 0.577 |
| | | 15.625 | 6.333 | 0.577 |
| | | 0 | 7.333 | 0.577 |

We claim:

1. A method of inhibiting sulfide production comprising:
(i) providing a composition comprising at least one compound having structure 1: 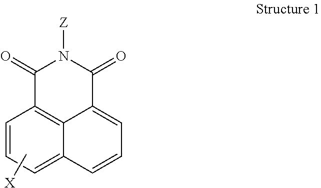
wherein, Z: C(O)NHOH or C(Y)(R),
Y: Hydrogen, Ce aromatic, Ce heteroaromatic, Ce aliphatic cyclic or alicyclic group, hetero group such as nitro, phosphate, hydroxyl,
R: Carbon 1-10 n or branched chain terminated with an N-hydroxycarboxamide, carboxylic acid, alcohol or N-hydroxycarboxamide,
X: Hydrogen, OH, NH₂, halogen, carbon (n=1-3) linear or branched chain compound; and
(ii) contacting the composition with a sulfide producing bacteria, under anaerobic conditions, to inhibit sulfide production.

2. The method of claim 1 wherein, the composition comprises the compound:

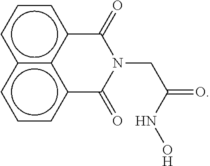

3. The method of claim 1 wherein, the composition comprises the compound:

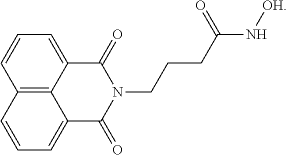 [SMILES: O=C(CCCN1C(=O)c2cccc3cccc(c23)C1=O)NO]

4. The method of claim 1 wherein, the composition inhibits sulfide production in oil and gas downhole application, subterranean hydrocarbon containing formation, functional fluids, oil and gas reservoirs and production systems, oil and gas transportation and storage systems, mining, industrial extraction of metals, cooling and heating systems, paper and pulp mills, membrane and filtration systems, material preservation, gas or liquid production, waste-water process, farming or slaughter house, land-fill, sewage collection system, municipality waste-water plant, coking coal process, or biofuel process.

5. A method of inhibiting sulfide production comprising:
(i) providing a composition comprising at least one compound having structure 2: 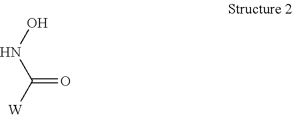
wherein, W: Hydrogen, carbon (n=1-10) linear or branched chain compound that is optionally terminated with a hydroxyamide, carboxylic acid, alcohol or N-hydroxycarboxamide, C₆ aromatic, C₆ heteroaromatic, C₆ aliphatic cyclic or alicyclic group; and
(ii) contacting the composition with a sulfide producing bacteria, under anaerobic conditions, to inhibit the sulfide production.

6. The method of claim 5 wherein, the composition comprises the compound:

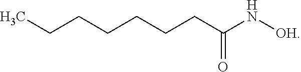

7. The method of claim 5 wherein, the composition inhibits sulfide production in oil and gas downhole application, subterranean hydrocarbon containing formation, functional fluids, oil and gas reservoirs and production systems, oil and gas transportation and storage systems, mining, industrial extraction of metals, cooling and heating systems, paper and pulp mills, membrane and filtration systems, material preservation, gas or liquid production, waste-water process, farming or slaughter house, land-fill, sewage collection system, municipality waste-water plant, coking coal process, or biofuel process.
